(19) United States
(12) Patent Application Publication
(10) Pub. No.: US 2002/0086066 A1
(43) Pub. Date: Jul. 4, 2002

(54) Skin cleaning composition and improved method for cleaning the face and body

(76) Inventors: Takeshi Fujimoto, Tokyo (JP); Hidenori Yokoi, Tokyo (JP)

Correspondence Address: LAFF, WHITESEL & SARET, LTD., 401 North Michigan Avenue, Chicago, IL, 60611, US

(21) Appl. No.: 09/751,133

(22) Filed: Dec. 29, 2000

(51) Int. Cl. A61K 35/78

(52) U.S. Cl. 424/725.1

(57) ABSTRACT

An improved skin cleaning composition made by introducing about 0.7% to about 2.0% by weight gas-activated charcoal to a base skin cleaning composition.

### BACKGROUND OF THE INVENTION

This invention relates generally to a skin cleaning composition for use on the human body and, more particularly, to a charcoal-containing skin cleaning composition which is gentle yet effective in cleaning and protecting the skin, and is especially useful as a facial cleanser. The invention also relates to a method of cleaning the face and body using this improved skin cleaning composition.

Many materials are available for cleansing the skin. These range from relatively pure metallic salts of fatty acids, to complex mixtures of various surfactants combined with a variety of other materials including medicinal agents, oils, alcohols, glycerin, vitamins, minerals, preservatives, organic and inorganic materials of speculative value, fragrances and coloring agents. These known cleaning materials vary in their effectiveness, both in terms of their penetration of the skin and their cleansing action, and in terms of their other effects on the skin such as causing or reducing wrinkling, causing or controlling drying, and accelerating or slowing the effects of aging. Generally, the more effective the known materials are in cleansing, the harsher they are on the skin. For example, many popular and effective skin cleaning materials cause significant drying and wrinkling, allergic reactions and other deleterious effects.

There is thus a need for a skin cleaning composition which is effective in cleaning, yet gentle to the skin. There is also a need for a skin cleaning composition which has these properties and, in addition, protects and preserves the skin, supplies antioxidants and minerals to the skin, and requires minimal amounts of preservatives which may cause allergic reactions.

### SUMMARY OF THE INVENTION

It is therefore an object of the present invention to provide a skin cleaning composition that is effective in cleaning, yet gentle to the skin and is especially useful in cleansing the face.

It is another object of the present invention to provide a skin cleaning composition that enhances the penetration of the water used along with the cleaning composition.

It is a further object of the present invention to provide a skin cleaning composition that helps preserve the skin by promoting moisture retention, promoting blood circulation, and reducing wrinkling.

It is yet another object of the present invention to provide a charcoal-containing skin cleaning composition that supplies antioxidants and minerals to the skin while absorbing oils and toxins from the skin into the porous surface of the charcoal.

Another object of the present invention is to provide a cleaning composition that includes a moisturizer, and which facilitates penetration of that moisturizer into the pores of the skin.

It is a still further object of the present invention to provide a cleaning composition that absorbs odors from drain lines into which the cleaning composition is flushed after use.

Finally, it is an object of the present invention to provide a skin cleaning composition that requires only minimal amounts of preservative.

In accordance with the present invention, these and other objects of the present invention will be achieved with an improved cleaning composition generally comprising a conventional skin cleaning composition to which about 0.7% to 2.0% by weight charcoal has been added.

### DETAILED DESCRIPTION OF A PREFERRED EMBODIMENT

The present inventors have discovered that charcoal, when added to conventional skin-cleaning compositions, significantly enhances the effectiveness of the skin-cleaning compositions in adsorbing dirt and dead skin cells trapped in the pores of the skin. It is believed that this results from the enhanced ability of the water used along with the cleaning compositions to penetrate the skin. Indeed, it has been found that the addition of charcoal to conventional skin cleaning compositions at the described levels may also help preserve the skin by promoting moisture retention, promoting blood circulation, and reducing wrinkling. A side benefit of the present invention is that the charcoal present in the cleaning composition enters the drain line after the rinse, absorbing odors from the drain line and reducing pollution.

The key ingredient of the present invention is charcoal made from wood by conventional processes, preferably medicinal charcoal. Medicinal charcoal is preferred because it is substantially free from potentially harmful contaminants. In many countries such medicinal charcoal is available in government-certified form as being substantially free from potentially harmful contaminants.

It is particularly preferred in the practice of this invention that the charcoal be made from sawdust from wood from Japanese cedar (cryptomeria) or Japanese cypress (white cedar) trees. These trees produce sawdust having a particularly large relative surface area and a highly porous surface. Other types of wood may be used in making the charcoal, but are less desirable.

The medicinal charcoal used in the present invention must be gas-activated. Gas activation employs oxygen, carbon dioxide, air, and water vapor as activation agents. Heat is applied externally in the activation process, and the activation agents are intermittently introduced into the furnace in which the wood is being heated to charcoal. Alternatively, an internal heating method can be used in which an activation temperature of about 800°-1000° C. is obtained using heated gases and by combustion of some of the sawdust used in making the activated charcoal. The activated material is circulated through the furnace and collected when ready. Although any conventional process for making gas-activated charcoal may be used, it is preferred in the practice of the present invention that charcoal made by the internal heating method be used, since the final activated material is most easily kept free of impurities.

The medicinal charcoal used in the invention must be in a particle form when added to the skin-cleaning composition. This particle size must be such that about 70%-80% of the particles would be removed using a 320 mesh per inch filter. The particle size is typically about 3 to 5 micrometers in diameter, but it may be as small as about 0.223 micrometers in diameter. When charcoal of particle sizes below 0.223 micrometers in diameter is used, it is difficult to get the cleaning composition to foam without rigorous scrubbing, which is generally not desirable where minimal abrasion of the skin is to be achieved. The pores on the surface of the charcoal particles must be from about 5 to 500 angstroms in diameter, and the surface area to mass ratio of the charcoal particles must be between about 1400 m² to about 3000 m² per gram.

The medicinal charcoal provides carbon atoms having a large number of active electrons, which are readily contributed to the surrounding atoms. It is believed that since the electron density of the charcoal is higher than that of the human body, the electrons flow from the areas of high density in the charcoal to areas of low electron density on the skin. Undisturbed atoms are composed of an equal number of positively charged protons in the nucleus, and negatively charged electrons orbiting the nucleus. This produces an overall neutral charge in the atom. When electrons leave the atom, by definition, the atom is in an oxidized state. The molecules in living cells are held together by chemical bonds forged by electrons. When the number of electrons is deficient, the molecules decompose, that is, the living cells are in an unhealthy oxidized state. Thus, the application of the charcoal with its excess electrons prevents this oxidation, keeping the cells healthy and vibrant. Indeed, it is believed that the cleaning composition of the present invention prevents aging of the skin by coating the skin surface with a protective layer of high electron density which inhibits oxidation.

The skin cleaning composition of the present invention should contain from about 0.7% by weight to about 2.0% by weight medicinal charcoal, based on the weight of the rest of the cleaning composition. When it is desired to provide the cleaning composition in a hardened bar form, it is preferred that charcoal at the lower end of the above described particle size range be used. On the other hand, when it is desired that the cleaning composition be provided in liquid form, charcoal of particle sizes anywhere in this range may be used.

Preferably, the conventional or base cleaning composition used in formulating the improved skin cleaning composition of the invention will be a soap base derived from plant oils. Most preferably, a combination of coconut oil and palm oil will be used. In a preferred embodiment, a combination of from about 20% to 35% by weight coconut oil and about 65% to 85% by weight palm oil will be used. These and other plant oils are preferred because they are gentle on the skin, and cause minimal allergic reactions and other adverse effects. Also, although not as desirable, animal oils and synthetic surfactants may be used in the cleaning composition.

The charcoal-containing cleaning composition of the present invention optimally may include a moisturizing agent. A preferred moisturizing agent in accordance with the present invention is sodium hydraluronic acid, which has been found to be particularly effective in promoting moisture retention while reducing skin wrinkling, when used in the present charcoal-containing composition. The cleaning composition of the present invention preferably will not include alcohols.

It is also desirable that the charcoal-containing cleaning composition include a stabilizer, which helps maintain the ingredients suspended and well distributed in the composition. A particularly preferred stabilizer is 1,2-pentanediol, which serves as a stabilizer as well as a moisturizer and preservative. The 1,2-pentanediol may be present at a level from about 0.5% to 1.5% by weight.

At least some of the charcoal used in making the composition is preferably steeped in water filtered through an ion-exchange membrane. The water is preferably maintained at room temperature and the charcoal is steeped in it for an extended period of time, preferably at least several minutes, and then added to the composition as a suspension. This steeping step is believed to partially dissolve the charcoal, increasing its antioxidant effects, increasing the mineral concentration in the water, and promoting cluster decomposition. As cluster decomposition progresses, the water's ability to permeate the skin is greatly improved. Additional charcoal may then be combined with the steeped charcoal which is then added to further increase the antioxidant concentrations, prevent the reformation of clusters, and extend the shelf life of the product.

When the material is to be hardened into bar form, preferably the bars may be in sizes as small as about 3 cm×5.5 cm×0.8 cm to as large as about 10 cm×7 cm×3 cm. The bars are made by heating the composition to about 40° C., pouring it into a mold corresponding to the shape and size of the desired bar, and allowing the material to dry and harden in the mold.

It is believed that the foregoing has made apparent many modifications in the described cleaning composition which will remain within the ambit of the invention disclosed. This invention, therefore, is only to be limited by the scope of the claims.

What we claim is:

1. An improved skin cleaning composition comprising a base skin cleaning composition to which about 0.7% to 2.0% by weight gas-activated charcoal has been added.

2. The improved skin cleaning composition of claim 1 in which the charcoal is medicinal charcoal.

3. The improved skin cleaning composition of claim 1 in which the charcoal is made from sawdust from Japanese cedar or Japanese cypress trees.

4. The improved skin cleaning composition of claim 1 in which the charcoal has a particle size of about 3 to 5 micrometers in diameter.

5. The improved skin cleaning composition of claim 1 in which the charcoal has a particle size of greater than about 0.223 micrometers in diameter.

6. The improved skin cleaning composition of claim 1 in which the pores on the surface of the charcoal are from about 5 to 500 angstroms in diameter, and the surface area to mass ratio of the charcoal particles is between 1400 m² and 3000 m² per gram.

7. The improved skin cleaning composition of claim 1 in which the base cleaning composition is derived from plant oils.

8. The improved skin cleaning composition of claim 7 in which the base cleaning composition includes from about 20% to 35% by weight coconut oil, and about 65% to 85% by weight palm oil.

9. The improved skin cleaning composition of claim 1 including sodium hydraluronic acid at a level sufficient to promote moisture retention while reducing skin wrinkling.

10. The improved skin cleaning composition of claim 1 including from about 0.5% to 1.5% by weight 1,2-pentanediol.

11. The improved skin cleaning composition of claim 1 in which the charcoal is steeped in water filtered though an ion-exchange membrane before being added to the base skin cleaning composition.

12. A method of improving a base skin cleaning composition comprising adding about 0.7% to 2.0% by weight gas-activated charcoal to the skin cleaning composition.

13. An improved skin cleaning composition comprising a base skin cleaning composition to which about 0.7% to 2.0% by weight gas-activated medicinal charcoal having a particle size of greater than about 0.223 micrometers in diameter has been added.

14. The improved skin cleaning composition of claim 13 in which the pores on the surface of the charcoal are from about 5 to 500 angstroms in diameter, and the surface area to mass ratio of the charcoal particles is between 1400 m² and 3000 m² per gram.

15. The improved skin cleaning composition of claim 13 in which the base cleaning composition is derived from plant oils.

16. The improved skin cleaning composition of claim 13 including heating the composition to about 40° C., and then pouring it into a mold corresponding to the shape and size of the desired bar, and allowing the material to dry and harden in the mold.
